(19) United States
(12) Patent Application Publication
(10) Pub. No.: US 2026/0294315 A1
(43) Pub. Date: Oct. 1, 2026

(54) METHOD, PROGRAM, AND DEVICE FOR DIAGNOSING HEART DISEASE ON BASIS OF ELECTROCARDIOGRAM SIGNAL

(71) Applicant: Medical AI Co., Ltd., Seoul (KR)

(72) Inventor: Joonmyoung Kwon, Seoul (KR)

(21) Appl. No.: 19/479,292

(22) PCT Filed: May 23, 2024
(86) PCT No.: PCT/KR2024/006998

§ 371 Date: Oct. 27, 2025

(30) Foreign Application Priority Data
May 23, 2023 (KR) 10-2023-0066571
May 23, 2024 (KR) 10-2024-0067060

(51) Int. Cl. A61B 5/346; A61B 5/00; G16H 50/20

(52) U.S. Cl. CPC A61B 5/346; A61B 5/7267; G16H 50/20

(57) ABSTRACT

The present disclosure provides a method, program, and device for diagnosing heart disease based on an electrocardiogram signal. According to one embodiment of the present disclosure, there is disclosed a method of diagnosing heart disease based on an electrocardiogram signal, the method being performed by a computing device including at least one processor, the method including: acquiring an electrocardiogram signal for a user; and diagnosing heart disease of the user based on the acquired electrocardiogram signal; wherein the diagnosing includes: computing a first score corresponding to the heart disease of the user by inputting the acquired electrocardiogram signal to a pre-trained neural network model; determining whether to additionally diagnose the heart disease of the user based on the computed first score, and reacquiring an electrocardiogram signal for the user; and additionally diagnosing the heart disease of the user based on the reacquired electrocardiogram signal.

### TECHNICAL FIELD

The present disclosure relates to deep learning technology in the medical field, and more particularly, to a method, program, and device for acquiring a user-customized neural network model that identifies bio-information.

### BACKGROUND ART

With the recent development of information and communication technology, deep learning technology is being utilized in various fields. In particular, deep learning technology is also being used in a variety of ways in the medical field, which has traditionally relied on specialized and limited personnel such as doctors, researchers, etc. to diagnose diseases of patients. For example, an example thereof is a case where the status or disease of a patient is analyzed by inputting the bio-data, acquired from the patient in real time, to a pre-trained deep learning model.

Meanwhile, myocardial infarction is the most common cause of sudden death, and the number of patients with myocardial infarction is increasing every year. Since the early detection and treatment of myocardial infarction can prevent death and disability, extensive research is being conducted on the early diagnosis and prediction of myocardial infarction. In connection with this, a method that measures electrocardiogram, displays the measured electrocardiogram signal in the form of a graph, and determines the presence or absence of myocardial infarction in the heart of a patient by using the graph is widely used for early diagnosis of myocardial infarction.

Recently, it has become possible to measure electrocardiograms of users by using portable electronic devices, such as smartwatches, without the need for specialized medical equipment. There have been proposed various methods that analyze an electrocardiogram of a user and diagnose and predict disease, such as myocardial infarction, of the user by using deep learning technology without the need for a medical professional. However, the diagnostic methods using deep learning often suffer from the problem of reduced accuracy depending on the status of a user.

### DISCLOSURE

### Technical Problem

The present disclosure has been conceived in response to the above-described background art, and an object of the present disclosure is to provide a method, program, and device for diagnosing heart disease based on an electrocardiogram signal.

However, the objects to be achieved in the present disclosure are not limited to the object mentioned above, and other objects not mentioned may be clearly understood based on the following description.

### Technical Solution

According to one embodiment of the present disclosure for achieving the above-described object, there is disclosed a method of diagnosing heart disease based on an electrocardiogram signal, the method being performed by a computing device including at least one processor, the method including: acquiring an electrocardiogram signal for a user; and diagnosing heart disease of the user based on the acquired electrocardiogram signal; wherein the diagnosing includes: computing a first score corresponding to the heart disease of the user by inputting the acquired electrocardiogram signal to a pre-trained neural network model; determining whether to additionally diagnose the heart disease of the user based on the computed first score, and reacquiring an electrocardiogram signal for the user; and additionally diagnosing the heart disease of the user based on the reacquired electrocardiogram signal.

Alternatively, the reacquiring an electrocardiogram signal for the user includes, when the computed first score is identified as being within a preset range, determining to perform an additional diagnosis for the heart disease of the user.

Alternatively, the method further includes acquiring status information of the user, and the reacquiring an electrocardiogram signal for the user includes, when an additional diagnosis for the heart disease of the user is determined to be performed, determining whether to reacquire an electrocardiogram signal for the user based on the acquired status information.

Alternatively, the status information includes medication information of the user, and the determining whether to reacquire an electrocardiogram signal includes, when the electrocardiogram signal is identified as acquired while the user had not taken a medication and then the user is identified as having taken the medication based on medication information of the user, determining to reacquire the electrocardiogram signal.

Alternatively, the status information includes medication information of the user, and the determining whether to reacquire an electrocardiogram signal includes, when the electrocardiogram signal is identified as acquired immediately after the user had taken a medication based on the medication information of the user, determining to reacquire the electrocardiogram signal after a preset period has elapsed since the user took the medication.

Alternatively, the status information includes disease information including at least one of palpitations, dyspnea, fatigue, and chest pain of the user, and the determining whether to reacquire an electrocardiogram signal includes, when the user is identified as having a disease based on the disease information of the user, determining to reacquire the electrocardiogram signal.

Alternatively, the status information includes activity information of the user, and the determining whether to reacquire an electrocardiogram signal includes, when a preset period has elapsed since the user began activity after waking up based on the activity information, determining to reacquire the electrocardiogram signal.

Alternatively, the status information includes exercise information of the user, and the determining whether to reacquire an electrocardiogram signal includes, when the user is identified as being performing a preset exercise, as having completed the preset exercise, or as having been in a resting state for a preset period since the completion of the preset exercise based on the exercise information, determining to reacquire the electrocardiogram signal.

Alternatively, the status information includes meal information of the user, and the determining whether to reacquire an electrocardiogram signal includes, when the user is identified as having just finished eating or a preset period is identified as having elapsed since the user finished eating based on the meal information, determining to reacquire the electrocardiogram signal.

Alternatively, the status information includes pulse rate of the user, and the determining whether to reacquire an electrocardiogram signal includes determining to reacquire the electrocardiogram signal based on the pulse rate.

Alternatively, the reacquiring an electrocardiogram signal for the user includes, when an additional diagnosis for heart disease of the user is determined to be performed, repeatedly reacquiring an electrocardiogram signal for the user.

Alternatively, the additionally diagnosing the heart disease of the user includes, each time an electrocardiogram signal for the user is reacquired, repeatedly additionally diagnosing the heart disease of the user based on the reacquired electrocardiogram signal and providing an additional diagnosis result.

Alternatively, the additionally diagnosing the heart disease of the user includes: computing a plurality of second scores corresponding to the heart disease of the user by inputting a plurality of repeatedly reacquired electrocardiogram signals to the pre-trained neural network model; and additionally diagnosing the heart disease of the user based on the plurality of second scores.

Alternatively, the additionally diagnosing the heart disease of the user includes: generating a first electrocardiogram signal by using the average bits of the plurality of repeatedly reacquired electrocardiogram signals; computing a second score corresponding to the heart disease of the user by inputting the generated first electrocardiogram signal to the pre-trained neural network model; and additionally diagnosing the heart disease of the user based on the second score.

Alternatively, the repeatedly reacquiring includes monitoring status information of the user, determining a reacquisition time for an electrocardiogram signal for the user based on the status information, and repeatedly reacquiring an electrocardiogram signal for the user based on the determined reacquisition time.

Alternatively, the reacquiring an electrocardiogram signal for the user includes: when the computed first score is identified as being within a preset range, determining whether there is a factor influencing the computed first score by analyzing the status information of the user; and, when it is determined that there is the factor, determining to perform an additional diagnosis for the heart disease of the user.

Alternatively, the reacquiring includes, when an additional diagnosis for the heart disease of the user is determined to be performed, repeatedly reacquiring an electrocardiogram signal for the user at a preset time interval.

Alternatively, the reacquiring an electrocardiogram signal for the user at a preset time interval includes, when the user is identified as having undergone surgery within a preset period, changing the preset time interval from a first interval to a second interval shorter than the first interval, and reacquiring an electrocardiogram signal for the user at the second interval.

According to one embodiment of the present disclosure for achieving the above-described object, there is disclosed a computer program stored in a computer-readable storage medium, the computer program causing operations for diagnosing heart disease based on an electrocardiogram signal to be performed when executed by at least one processor, wherein the operations include operations of: acquiring an electrocardiogram signal for a user; and diagnosing heart disease of the user based on the acquired electrocardiogram signal; wherein the operation of diagnosing heart disease of the user based on the acquired electrocardiogram signal includes operations of: computing a first score corresponding to the heart disease of the user by inputting the acquired electrocardiogram signal to a pre-trained neural network model; determining whether to additionally diagnose the heart disease of the user based on the computed first score, and reacquiring an electrocardiogram signal for the user; and additionally diagnosing the heart disease of the user based on the reacquired electrocardiogram signal.

According to one embodiment of the present disclosure for achieving the above-described object, there is disclosed a computing device for acquiring a user-customized neural network model that identifies bio-information, the computing device including: memory including program codes; a sensing unit configured to acquire an electrocardiogram signal of a user; and a processor configured to acquire an electrocardiogram signal for the user via the sensing unit, and diagnose heart disease of the user based on the acquired electrocardiogram signal; wherein the processor computes a first score corresponding to the heart disease of the user by inputting the acquired electrocardiogram signal to a pre-trained neural network model, determines whether to additionally diagnose the heart disease of the user based on the computed first score, reacquires an electrocardiogram signal for the user, and additionally diagnoses the heart disease of the user based on the reacquired electrocardiogram signal.

### Advantageous Effects

The method of diagnosing heart disease based on an electrocardiogram signal according to one embodiment of the present disclosure allows a user to determine the status of heart disease in real time without the assistance of a specialized medical institution or medical professional.

### DESCRIPTION OF DRAWINGS

FIG. 1 is an exemplary diagram showing a method of predicting heart disease based on an electrocardiogram signal of a user according to one embodiment of the present disclosure;

FIG. 2 is a schematic block diagram of a computing device according to one embodiment of the present disclosure;

FIG. 3 is a flowchart schematically showing a method of diagnosing heart disease of a user based on an electrocardiogram signal according to one embodiment of the present disclosure;

FIG. 4 is a flowchart schematically showing a method of repeatedly diagnosing heart disease of a user based on an electrocardiogram signal according to one embodiment of the present disclosure;

FIG. 5 is a flowchart schematically showing a method of repeatedly diagnosing heart disease of a user based on status information of the user according to one embodiment of the present disclosure; and

FIG. 6 is a block diagram of a computing device according to another embodiment of the present disclosure.

### MODE FOR INVENTION

Embodiments of the present disclosure will be described in detail below with reference to the accompanying drawings so that those having ordinary skill in the art of the present disclosure (hereinafter, those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Furthermore, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “N-th (N is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

The term “acquiring” used herein may be understood to mean not only receiving data over a wired/wireless communication interface connecting with an external device or a system, but also generating data in an on-device form.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Furthermore, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “model” used herein may be understood as a system implemented using mathematical concepts and language to solve a specific problem, a set of software units intended to solve a specific problem, or an abstract model for a process intended to solve a specific problem. For example, a neural network “model” may refer to an overall system implemented as a neural network that is provided with problem-solving capabilities through training. In this case, the neural network may be provided with problem-solving capabilities by optimizing parameters connecting nodes or neurons through training. The neural network “model” may include a single neural network, or a neural network set in which multiple neural networks are combined together.

The “data” used herein may include an “image,” a signal, and the like. The term “image” used herein may refer to multidimensional data composed of discrete image elements. In other words, the “image” may be understood as a term referring to a digital representation of an object that is visible to the human eye. For example, the term “image” may refer to multidimensional data composed of elements corresponding to pixels in a two-dimensional image. The term “image” may refer to multidimensional data composed of elements corresponding to voxels in a three-dimensional image.

The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

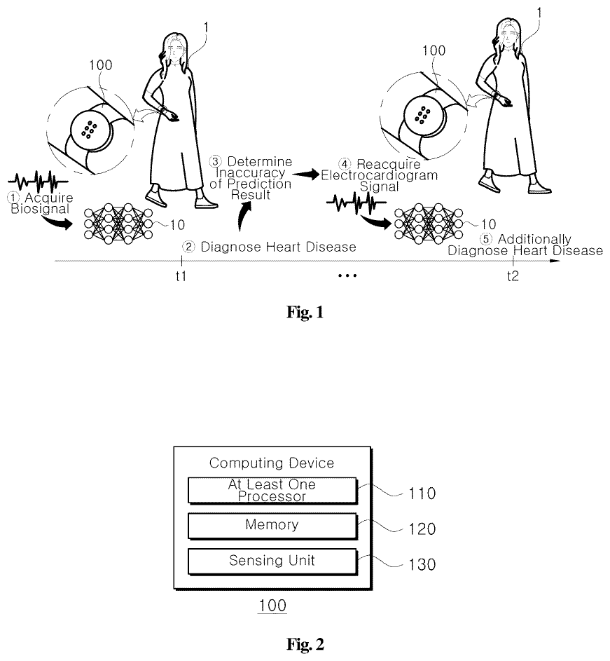

FIG. 1 is an exemplary diagram showing a method of predicting heart disease based on an electrocardiogram signal of a user according to one embodiment of the present disclosure.

A computing device 100 according to one embodiment of the present disclosure may be a hardware device or a part of a hardware device that performs the comprehensive processing and computation of data, or may be a software-based computing environment connected via a communication interface. For example, the computing device 100 may be a server that is a main agent for performing an intensive data processing function and sharing resources, or may be a client that shares resources through interaction with a server. Alternatively, the computing device 100 may be a cloud system in which multiple servers and clients comprehensively process data while interacting with each other. Since the above description is only one example related to the type of computing device 100, the type of computing device 100 may be configured in various manners within a range understandable to those skilled in the art based on the content of the present disclosure.

Referring to FIG. 1, a computing device 100 according to an embodiment of the present disclosure may be implemented as a smartwatch. However, the computing device 100 is not limited thereto, but may be implemented as various electronic devices such as a desktop, a laptop, a smartphone, a server device, a smart band, a smart ring, or the like. For the purpose of understanding the present disclosure, the following description will be given on the assumption that the computing device 100 is a smartwatch.

According to one embodiment of the present disclosure, the computing device 100 may acquire an electrocardiogram signal from a user 1. More specifically, the computing device 100 may acquire an electrocardiogram signal by detecting electrical signals generated from the heart of the user 1. Meanwhile, in addition to the electrocardiogram signal, the computing device 100 may acquire various bio-signals from the user 1. For example, the computing device 100 may acquire various types of bio-information, such as the pulse, body temperature, blood pressure, blood flow and/or the like of the user 1 by using various sensors.

According to one embodiment of the present disclosure, the computing device 100 may store a pre-trained neural network model 10. The neural network model 10 may be a model 10 trained to predict a potential heart disease of the user 1 based on an electrocardiogram signal of the user 1. The neural network model 10 may be pre-trained to predict heart disease based on an input electrocardiogram signal based on training data, which includes a plurality of electrocardiogram signals and heart disease information (e.g., the presence/absence of a heart disease, the degree of heart disease, and/or the like) matching each electrocardiogram signal. In this case, the plurality of electrocardiogram signals included in the training data may each include feature information (e.g., feature points shown on the electrocardiogram signal) associated with heart disease. Through this, when an electrocardiogram signal is input, the neural network model 10 is trained to extract feature information corresponding to heart disease from the electrocardiogram signal and predict the likelihood of heart disease based on the extracted feature information. The neural network model 10 may be pre-trained to score the likelihood of heart disease. In this case, heart disease may include various diseases and symptoms that manifest in the heart, such as heart failure or the like.

Furthermore, the pre-trained neural network model 10 may include a plurality of neural network models 10 trained to predict a potential disease of the user 1 based on various types of bio-information acquirable from the computing device 100, as well as an electrocardiogram signal. In this case, each of the neural network models 10 may be pre-trained using disease information (e.g., disease presence/absence information) to be predicted or identified as training data based on bio-information and biological information matching the bio-information.

The neural network model 10 may be implemented as a multi-layer perceptron (MLP), a convolutional neural network (CNN), a recurrent neural network (RNN), a generative adversarial network, or the like. In particular, the neural network model 10 may include a plurality of residual blocks configured to extract latent features of an input electrocardiogram signal, and a classifier configured to classify a heart disease based on the extracted latent features.

Meanwhile, referring to FIG. 1, the computing device 100 may repeatedly acquire an electrocardiogram signal of the user 1 and predict heart disease in order to increase the accuracy of predicting heart disease of the user 1 based on the electrocardiogram signal. More specifically, when the computing device 100 acquires an electrocardiogram signal at time t1 and predicts heart disease of the user 1 in response to a request from the user 1 (or according to a preset condition), it may reacquire an electrocardiogram signal at time t2 and then predict heart disease of the user 1 again. In this case, the computing device 100 may reacquire the electrocardiogram signal at time t2 and predict heart disease of the user 1 without a request from the user 1. Furthermore, the computing device 100 may additionally diagnose heart disease of the user 1 based on a prediction result for the heart disease of the user 1 at time t2.

In particular, when it is determined that the prediction result for the heart disease based on the electrocardiogram signal is likely to be inaccurate, the computing device 100 may reacquire an electrocardiogram signal and re-predict heart disease of the user 1 based on the reacquired electrocardiogram signal. In this case, the fact that the prediction result for the heart disease based on the electrocardiogram signal is likely to be inaccurate may mean that the prediction result is ambiguous or not highly accurate.

In this case, the computing device 100 may repeatedly perform additional diagnosis. More specifically, the computing device 100 may repeatedly reacquire an electrocardiogram signal, and may repeatedly diagnose heart disease of the user 1 based on the reacquired electrocardiogram signal each time the electrocardiogram signal is reacquired. Furthermore, the computing device 100 may provide a diagnosis result to the user 1 each time the additional diagnosis is performed.

In the following description, one embodiment of the present disclosure will be described in detail with reference to FIGS. 2 to 6.

FIG. 2 is a schematic block diagram of a computing device 100 according to one embodiment of the present disclosure.

Referring to FIG. 2, the computing device 100 according to one embodiment of the present disclosure may include at least one processor (hereinafter, the “processor”) 110, memory 120, and a sensing unit 130. However, FIG. 1 is merely an example, and the computing device 100 may further include other components for implementing a computing environment. Furthermore, only some of the disclosed components may be included in the computing device 100.

The processor 110 according to one embodiment of the present disclosure may be understood as a constituent unit including hardware and/or software for performing computing operations. For example, the processor 110 may read a computer program and perform data processing for machine learning. The processor 110 may process operation processes such as the processing of input data for machine learning, the extraction of features for machine learning, and the computation of errors based on backpropagation. The processor 110 for performing such data processing may include a central processing unit (CPU), a general purpose graphics processing unit (GPGPU), a tensor processing unit (TPU), an application specific integrated circuit (ASIC), and a field programmable gate array (FPGA). Since the types of processor 110 described above are only examples, the type of processor 110 may be configured in various manners within a range understandable to those skilled in the art based on the content of the present disclosure.

The processor 110 is electrically connected to other components (i.e., the memory 120, network interface 240, and sensing unit 130) of the computing device 100, and controls the overall operation of the computing device 100.

The memory 120 according to one embodiment of the present disclosure may be understood as a constituent unit including hardware and/or software for storing and managing data that is processed in the computing device 100. That is, the memory 120 may store any type of data generated or determined by the processor 110 and any type of data received by the communication interface. For example, the memory 120 may include at least one type of storage medium of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 120 may include a database system that controls and manages data in a predetermined system. Since the types of memory 120 described above are only examples, the type of memory 120 may be configured in various manners within a range understandable to those skilled in the art based on the content of the present disclosure.

The memory 120 may structure, organize, and manage the data required for the processor 110 to perform operations, combinations of the data, and program codes executable by the processor 110. For example, the memory 120 may store a neural network model 10 trained to predict heart disease based on an electrocardiogram signal. Furthermore, the memory 120 may include program codes configured to operate the acquired neural network model 10 to perform training, and training data (for example, a plurality of electrocardiogram signals and a plurality of pieces of heart disease information matching the respective electrocardiogram signals). Furthermore, the memory 120 may store program codes configured to operate the neural network model 10 to receive electrocardiogram signals and perform inference in accordance with the purpose of use of the computing device 100, and processed data generated as the program codes are executed.

The sensing unit 130 may acquire an electrocardiogram signal for the user 1. As an example, the sensing unit 130 may include at least one electrode. In this case, the processor 110 may acquire an electrocardiogram signal for the user 1 via the at least one electrode. Furthermore, the sensing unit 130 may include an image sensor or an optical sensor. In this case, the processor 110 may acquire bio-information, such as a photoplethysmogram signal, via the image sensor (or the optical sensor).

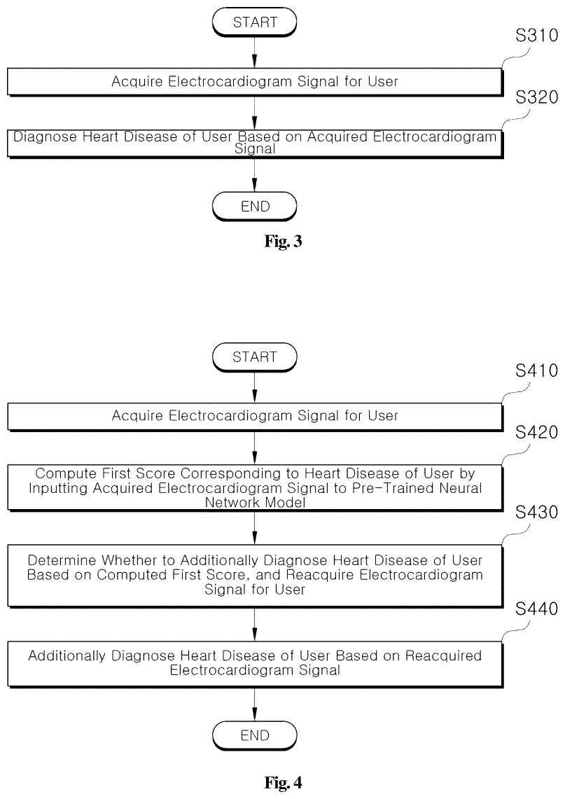

FIG. 3 is a flowchart schematically showing a method of diagnosing heart disease of a user 1 based on an electrocardiogram signal according to one embodiment of the present disclosure.

Referring to FIG. 3, according to one embodiment of the present disclosure, the processor 110 acquires an electrocardiogram signal for the user 1 in step S310. In this case, step S310 may also be referred to as an initial electrocardiogram signal acquisition step in that it is distinct from the step of reacquiring an electrocardiogram signal.

More specifically, the processor 110 may receive a request to diagnose heart disease from the user 1 via the interface or communication interface of the computing device 100. In this case, when the processor 110 receives a request to diagnose heart disease from the user 1, it may acquire an electrocardiogram signal for the user 1. Alternatively, the processor 110 may acquire an electrocardiogram signal according to a preset condition (e.g., time, interval, and/or the like). However, the acquisition of an electrocardiogram signal is not limited thereto, but the processor 110 may continuously acquire an electrocardiogram signal while the user 1 wears the computing device 100.

The processor 110 may acquire an electrocardiogram signal by detecting electrical signals generated from the heart of the user 1 via the sensing unit 130.

Then, the processor 110 diagnoses the heart disease of the user 1 based on the acquired electrocardiogram signal in step S320.

More specifically, when an electrocardiogram signal is acquired, the processor 110 may identify whether the user 1 has a heart disease based on the acquired electrocardiogram signal. Alternatively, the processor 110 may predict a heart disease of the user 1 based on the acquired electrocardiogram signal. That is, the processor 110 may identify, based on the electrocardiogram signal, whether the user 1 has a potential heart disease that is not yet manifested but is likely to develop in the future. In particular, the processor 110 may extract the potential characteristic information of a heart disease from the electrocardiogram signal and predict the heart disease.

To this end, the processor 110 may utilize the pre-trained neural network model 10. In this case, the pre-trained neural network model 10 may be a model 10 trained to identify the presence/absence of a heart disease of the user 1 based on an electrocardiogram signal or predict a heart disease of the user 1. Since the description of the invention given with reference to FIG. 1 applies equally to the pre-trained neural network model 10, a detailed description thereof will be omitted.

Meanwhile, the processor 110 may acquire a score corresponding to the heart disease by inputting the electrocardiogram signal to the pre-trained neural network model 10. That is, the pre-trained neural network model 10 may be trained to compute a score corresponding to the heart disease predicted based on the input electrocardiogram signal. As an example, the pre-trained neural network model 10 may be trained to compute a higher score as the likelihood of heart disease increases.

The processor 110 may acquire the score from the pre-trained model 10 and diagnose a heart disease of the user 1 based on the acquired score. More specifically, when the acquired score is equal to or higher than a preset value, the processor 110 may diagnose the user 1 as having a high likelihood of heart disease. For example, when the preset value is 10 and the heart disease is heart failure, the processor 110 may diagnose the user as having a high likelihood of heart failure when the score acquired from the pre-trained neural network model 10 is equal to or higher than 10.

Meanwhile, the processor 110 may output a diagnosis result. For example, the processor 110 may output the diagnosis result via the display or output interface of the computing device 100. Furthermore, the processor 110 may also output user (1) guidance information based on the diagnosis result. As for the above-described example again, when the likelihood of heart failure of the user 1 is identified as high based on the electrocardiogram signal, the processor 110 may output information alerting the user 1 of the risk of heart failure and information about a nearby hospital based on the location information of the user 1 via the display.

FIG. 4 is a flowchart schematically showing a method of repeatedly diagnosing heart disease of a user 1 based on an electrocardiogram signal according to one embodiment of the present disclosure. Step S410 shown in FIG. 4 may correspond to step S310 shown in FIG. 3.

Meanwhile, according to one embodiment of the present disclosure, the processor 110 may reacquire an electrocardiogram signal and re-diagnose heart disease based on the reacquired electrocardiogram signal to improve the accuracy of the diagnosis result of heart disease. To this end, the processor 110 may first determine whether the diagnosis result of heart disease is likely to be inaccurate. That is, the processor 110 may determine whether to diagnose heart disease based on the acquired electrocardiogram by determining whether the diagnosis result of the heart disease is likely to be inaccurate. Furthermore, when it is determined that the diagnosis result of the heart disease is likely to be inaccurate, the processor 110 may reacquire an electrocardiogram signal and re-diagnose heart disease.

Referring to FIG. 4, the processor 110 may compute a score corresponding to the heart disease of the user 1 by inputting the acquired electrocardiogram signal to the pre-trained neural network model 10 in step S420. In the following description, for ease of description, the score initially computed based on the electrocardiogram signal will be referred to as a first score.

Furthermore, the processor 110 may determine whether to additionally diagnose heart disease of the user 1 based on the computed first score and reacquire an electrocardiogram signal for the user in step S430. When an additional diagnosis for heart disease of the user 1 is determined to be performed, the processor 110 may repeatedly reacquire an electrocardiogram signal for the user 1.

According to one embodiment of the present disclosure, when the computed first score is identified as being within a preset range, the processor 110 may determine to perform an additional diagnosis for heart disease of the user 1. In contrast, when the computed first score is identified as being outside the preset range, the processor 110 may determine to diagnose heart disease of the user 1 based on the computed first score.

For example, in the case where the preset range spans from 9.5 to 10.5, when the first score is identified as 10, the processor 110 may determine to perform an additional diagnosis for heart disease of the user 1. That is, the processor 110 may determine to perform an additional diagnosis for heart disease based on the first score 10 by determining that the diagnosis result based on the first score 10 is likely to be inaccurate.

Furthermore, for example, in the case where the preset range spans from 9.5 to 10.5, when the first score is identified as 12, the processor 110 may determine not to perform an additional diagnosis of the user 1. That is, the processor 110 may determine that the diagnosis result based on the first score of 12 is accurate, and may determine to diagnose heart disease based on the first score of 12. In this case, the processor 110 may provide the user 1 with the diagnosis result of heart disease and guidance information corresponding to the diagnosis result.

Meanwhile, the preset range may be set based on the biological characteristics (age, weight, gender, and/or the like) of the user 1.

According to one embodiment of the present disclosure, when the computed first score is identified as being within the preset range, the processor 110 analyzes status information of the user 1 to determine whether there is any factor that influences the computed first score. When it is determined that there is a factor, the processor 110 may determine to perform an additional diagnosis for heart disease of the user 1.

More specifically, when the computed first score is identified as being within the preset range, the processor 110 determines that the diagnosis of heart disease based on the first score is likely to be inaccurate and analyzes a factor that influences the inaccuracy of the diagnosis. To this end, the processor 110 may utilize status information of the user 1.

The processor 110 may acquire status information of the user 1. The status information of the user 1 is information indicating the status of the user 1, such as the exercise status of the user 1 or the eating status of the user 1. Furthermore, the status information of the user 1 may further include bio-information (blood pressure, pulse, body temperature, blood flow, and/or the like) in addition to an electrocardiogram signal of the user 1.

When the computed first score is identified as being within the preset range, the processor 110 may determine the status of the user 1 by analyzing the acquired status information of the user 1. Furthermore, based on the status of the user 1, the processor 110 may determine whether there is any factor that influences the computed first score. For example, when an electrocardiogram signal is acquired and the user 1 is determined to be in an exercise state, the processor 110 may determine that an electrocardiogram of the user 1 is influenced by factors such as increased blood flow and body temperature due to the exercise of the user 1. That is, the processor 110 may determine that the increased blood flow and body temperature due to the exercise state of the user 1 influence the first score computed based on the electrocardiogram signal.

When it is determined that there is a factor that influences the first score, the processor 110 may determine to perform an additional diagnosis in addition to the diagnosis of heart disease using the first score. In particular, when a factor influencing the first score is identified as continuing to be present based on the status information of the user 1 (or when the status of the user 1 in which a factor influencing the first score occurs is identified as continuing), the processor 110 may determine to perform an additional diagnosis.

Furthermore, when the electrocardiogram signal is identified as containing a large amount of noise, the processor 110 may determine to perform an additional diagnosis for heart disease of the user 1 based on the electrocardiogram signal. More specifically, when the first score is outside the preset range and the noise contained in the electrocardiogram signal is equal to or higher than a preset value, the processor 110 may determine to perform a diagnose for heart disease of the user 1 based on the first score and then perform an additional diagnosis based on a reacquired electrocardiogram signal.

Furthermore, the processor 110 may determine to perform an additional diagnosis of heart disease based on an electrocardiogram signal by comparing the diagnosis result of heart disease based on another type of bio-information for the user 1 with the diagnosis result of heart disease based on an electrocardiogram signal. That is, the processor 110 may identify the first score as being outside the preset range, and may compare the diagnosis result of heart disease based on the first score with the diagnosis result of heart disease acquired based on another type of bio-information (e.g., an electroencephalogram, or the like) for the user 1. In this case, when the diagnosis results (i.e., the diagnosis result based on the electrocardiogram signal and the diagnosis result based on the other type of bio-information) are identified as being different from each other, the processor 110 may determine to reacquire an electrocardiogram signal and perform an additional diagnosis of heart disease.

When an additional diagnosis for heart disease of the user 1 is determined to be performed, the processor 110 may reacquire an electrocardiogram signal for the user 1. More specifically, when an additional diagnosis for heart disease of the user 1 is determined to be performed, the processor 110 may reacquire an electrocardiogram signal for the user 1 by using the sensing unit 130. In particular, when an additional diagnosis for heart disease of the user 1 is determined to be performed, the processor 110 may reacquire an electrocardiogram signal for the user 1, regardless of a request from the user 1 or a preset condition.

Furthermore, when an additional diagnosis for heart disease of the user 1 is determined to be performed, the processor 110 may output, via a display or output interface, a message indicating that the diagnosis result of heart disease may be inaccurate and a message requesting a re-request for a heart disease diagnosis after a preset time.

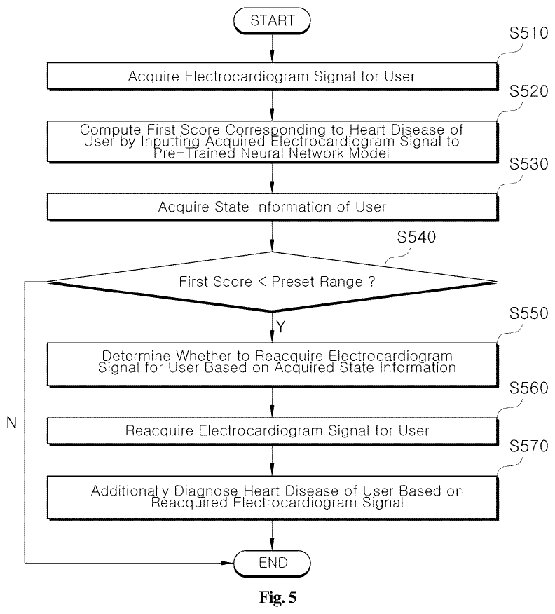

FIG. 5 is a flowchart schematically showing a method of repeatedly diagnosing heart disease of a user 1 based on status information of the user 1 according to one embodiment of the present disclosure. Steps S510, S520, and S570 shown in FIG. 5 may correspond to steps S410, S420, and S440 shown in FIG. 4.

Referring to FIG. 5, according to one embodiment of the present disclosure, the processor 110 may acquire status information of the user 1 in step S530. The status information of the user 1 may be acquired via the sensing unit 130 in real time. However, the acquisition of the status information is not limited thereto, but the status information of the user 1 may also be acquired through input via the communication interface or input interface of the computing device 100.

When an additional diagnosis for heart disease of the user 1 is determined to be performed by comparing the first score with the preset range, the processor 110 may determine whether to reacquire an electrocardiogram signal for the user 1 based on the status information acquired at the time at which the electrocardiogram signal was acquired in step S550. The status information of the user 1 is information representing the status of the user 1, such as the exercise status of the user 1 or the eating status of the user 1. Furthermore, the status information of the user 1 may further include bio-information (blood pressure, pulse, body temperature, blood flow, and/or the like) in addition to an electrocardiogram signal of the user 1.

In particular, when it is determined that there is no factor influencing the first score by monitoring status information of the user 1, the processor 110 may determine to reacquire an electrocardiogram signal for the user 1. That is, based on the status information, the processor 110 may determine the time at which an electrocardiogram signal is reacquired in order to compute a more accurate score.

According to one embodiment of the present disclosure, the status information may include medication information of the user 1. In this case, the medication information may be received from a smart medicine cabinet or identified based on the medication time set in the computing device 100. In this case, when the user 1 is identified as having taken a medication based on the medication information, the processor 110 may determine to reacquire an electrocardiogram signal.

More specifically, based on medication information, the processor 110 may determine that the medication status of the user 1 influences the electrocardiogram signal and the first score is inaccurately computed. Accordingly, when the user 1 is identified as having taken a medication based on the medication information, the processor 110 may determine to reacquire an electrocardiogram signal for the user 1. As an example, when the electrocardiogram signal for the user 1 is identified as having been acquired before the user took the medication and also the user is identified as then having taken the medication based on the medication information, the processor 110 may determine to reacquire an electrocardiogram signal. In this case, the processor 110 may compare the time at which the user 1 took the medication with the time at which the electrocardiogram signal for the user 1 was acquired based on the medication information.

Furthermore, when the electrocardiogram signal is identified as acquired immediately after the user had taken the medication based on medication information of the user 1, the processor 110 may determine to reacquire an electrocardiogram signal after a preset period has elapsed since the user took the medication. More specifically, the processor 110 may identify the time at which the user 1 took the medication based on the medication information of the user 1. In this case, when the time at which the electrocardiogram signal for the user 1 is identified as acquired was within a preset first period (e.g., two seconds) from the time at which the user 1 has taken the medication (i.e., when the electrocardiogram signal is identified as acquired immediately after the user 1 had taken the medication) based on the medication information, the processor 110 may determine to reacquire an electrocardiogram signal for the user 1 after a preset second period (e.g., ten minutes).

According to one embodiment of the present disclosure, the status information may include pulse rate information of the user 1. In this case, the pulse rate information may be acquired based on a photoplethysmogram (PPG) sensor included in the sensing unit 130 of the computing device 100. Furthermore, the processor 110 may determine whether to reacquire an electrocardiogram signal based on the pulse rate. More specifically, based on medication information, the processor 110 may determine that the electrocardiogram signal may be acquired differently depending on the pulse of the user 1 and the first score may be inaccurately computed based on the pulse rate. Accordingly, the processor 110 may determine whether to reacquire an electrocardiogram signal based on the pulse rate information. For example, when it is determined based on pulse rate information of the user 1 that the electrocardiogram signal was acquired while the pulse rate of the user 1 was unstable and then the pulse rate of the user 1 was stabilized, the processor 110 may determine to reacquire an electrocardiogram signal for the user 1. Alternatively, when the pulse rate of the user 1 changes rapidly or becomes irregular based on the pulse rate information of the user 1, the processor 110 may determine to reacquire an electrocardiogram signal for the user 1.

According to one embodiment of the present disclosure, the status information may include disease information of the user 1. In this case, the disease information may include at least one of palpitations, dyspnea, fatigue, and chest pain. However, the disease information is not limited thereto, but various diseases may be included in the status information. In this case, when the user 1 is identified as having a disease based on the disease information, the processor 110 may determine to reacquire an electrocardiogram signal. In this case, the disease information may be input by the user 1 via the user interface of the computing device 100, or may be acquired by identifying the occurrence of a disease by using pulse rate and/or the like.

According to one embodiment of the present disclosure, the status information may include activity information of the user 1. The activity information may include movement information of the user 1, which includes information about whether the user 1 is sleeping, whether the user 1 is awake, and whether the user 1 is active after waking up. In this case, when a preset third period (e.g., 30 minutes) is identified as having elapsed since the user began activity after waking up based on the activity information, the processor 110 may determine to reacquire an electrocardiogram signal. More specifically, when the preset third period is identified as having elapsed since the user 1 woke up and began activity based on the activity information, the processor 110 may determine to reacquire an electrocardiogram signal for the user 1. In particular, when an electrocardiogram signal of the user 1 is identified as acquired while the user 1 was sleeping based on the activity information, the processor 110 may determine to reacquire an electrocardiogram signal after the user 1 has woken up. Meanwhile, the activity information may be acquired by detecting the movement of the user 1 via the sensing unit 130, or may be input by the user 1 via the user interface of the computing device 100.

According to one embodiment of the present disclosure, the status information may include exercise information of the user 1. In this case, the exercise information includes information about the type of exercise performed by the user 1 and information about whether the user is progressing with the exercise. When the user is identified as being performing a preset exercise, has completed the preset exercise, or has been resting for a preset fourth period (e.g., 30 minutes) from the time at which the preset exercise ended based on the exercise information, the processor 110 may determine to reacquire an electrocardiogram signal. In this case, the preset exercise is classified as vigorous exercise. For example, the preset exercise includes running, mountain hiking, stair climbing, and the like. Meanwhile, the exercise information may be acquired by analyzing the movement of the user 1 detected by the sensing unit 130, or information about the type of exercise and information about the progress of the exercise may be input by the user 1 via the user interface of the computing device 100.

According to one embodiment of the present disclosure, the status information may include meal information of the user 1. In this case, the meal information may include information about whether the user 1 has eaten and information about the type of food. In this case, when the user is identified as having just finished eating or a preset period is identified as having elapsed since the user finished eating based on the meal information, the processor 110 may determine to reacquire an electrocardiogram signal. When the time is within a preset fifth period (e.g., five minutes) from the time at which the user finished eating, the processor 110 may determine that the user has just finished eating. Meanwhile, the meal information may be acquired based on an image captured via the camera of the computing device 100, or may be input by the user 1 via the user interface.

Meanwhile, according to one embodiment of the present disclosure, when an additional diagnosis for heart disease of the user 1 is determined to be performed, the processor 110 may reacquire an electrocardiogram signal for the user at a preset time interval. For example, when an additional diagnosis for heart disease of the user 1 is determined to be performed, the processor 110 may reacquire an electrocardiogram signal every day. That is, when an additional diagnosis for heart disease of the user 1 is determined to be performed, the processor 110 may reacquire an electrocardiogram signal once a day. However, the preset time interval is not limited thereto, but may be set to various values, such as five minutes, one week, or one month.

In this case, the processor 110 may change the preset time interval depending on the status of the user 1. In particular, when the user is identified as having undergone surgery within a preset six-hour period, the processor 110 may change the preset time interval to a shorter period. In this case, whether the user 1 has undergone surgery may be acquired as the status information of the user 1, and may be acquired via the communication interface of the computing device 100 or input by the user 1 via the user interface. For example, as additional diagnosis for heart disease of the user 1 is determined to be performed, the processor 110 may acquire status information of the user 1 while reacquiring an electrocardiogram signal once a day. In this case, when the user is identified as having undergone surgery within a week (a preset sixth period), based on the acquired status information, the processor 110 may change the reacquisition time interval from one day to a shorter six-hour period and reacquire an electrocardiogram signal of the user every six hours. Referring to FIG. 5, when an electrocardiogram signal for the user is determined to be reacquired based on the status information, the processor 110 may reacquire an electrocardiogram signal for the user via the sensing unit 130 in step S560.

Referring back to FIG. 4, the processor 110 may additionally diagnose heart disease of the user 1 based on the reacquired electrocardiogram signal in step S440.

More specifically, the processor 110 may reacquire a score corresponding to a heart disease by inputting the reacquired electrocardiogram signal to the pre-trained neural network model 10. Hereinafter, for ease of description of the present invention, a score recomputed based on a reacquired electrocardiogram signal will be referred to as a second score. The processor 110 may then re-diagnose heart disease of the user 1 based on the second score.

In this case, when the second score is within a preset range, the processor 110 may diagnose heart disease of the user 1 based on the second score, may then reacquire an electrocardiogram signal to calculate a score (i.e., a third score), and may further diagnose heart disease of the user 1 based on the third score.

Meanwhile, according to one embodiment of the present disclosure, when an additional diagnosis for heart disease of the user 1 is determined to be performed, the processor 110 may repeatedly reacquire an electrocardiogram signal for the user 1. That is, the processor 110 may reacquire an electrocardiogram signal for the user 1 multiple times, may perform an additional diagnosis for heart disease of the user 1 each time the electrocardiogram signal is reacquired, and may provide an additional diagnosis result. That is, the additional diagnosis result may be repeatedly provided.

In particular, the processor 110 may acquire status information of the user, may monitor status information of the user, and may determine a reacquisition time for an electrocardiogram signal of the user based on the status information. Furthermore, the processor 110 may repeatedly reacquire an electrocardiogram signal of the user based on the determined reacquisition time. The processor 110 may compute a plurality of second scores corresponding to heart disease of the user 1 by inputting a plurality of repeatedly reacquired electrocardiogram signals to the pre-trained neural network model 10, and may additionally diagnose heart disease of the user 1 based on the plurality of second scores. For example, the processor 110 may compute a third score based on the average, median, or most frequent value of the plurality of second scores, and may additionally diagnose heart disease of the user 1 based on the computed third score.

Meanwhile, according to one embodiment of the present disclosure, when an additional diagnosis for heart disease of the user 1 is determined to be performed, the processor 110 may repeatedly reacquire an electrocardiogram signal for the user 1, and may then generate a new electrocardiogram signal (a first electrocardiogram signal) by using the average bits of a plurality of reacquired electrocardiogram signals. That is, the processor 110 may identify the bits of the plurality of electrocardiogram signals and generate a first electrocardiogram signal having the average bits of the plurality of electrocardiogram signals. In this case, the first electrocardiogram signal differs from an electrocardiogram signal directly measured from the user 1 in that it is generated by the processor 110. Furthermore, the processor 110 may compute a second score corresponding to the heart disease of the user 1 by inputting the generated first electrocardiogram signal to the pre-trained neural network model 10, and may additionally diagnose heart disease of the user 1 based on the second score.

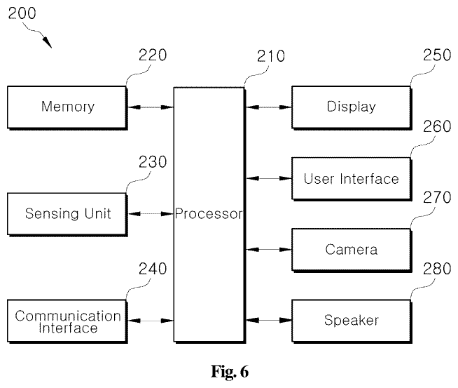

FIG. 6 is a block diagram of a computing device 200 according to another embodiment of the present disclosure.

Referring to FIG. 6, the computing device 200 according to the embodiment of the present disclosure includes a processor 210, memory 220, a sensing unit 230, a communication interface 240, a display 250, a user interface 260, a camera 270, and a speaker 280. Among the components shown in FIG. 6, the processor 210, memory 220, and sensing unit 230 shown in FIG. 6 correspond to the processor 110, memory 120, and sensing unit 130 of the computing device 100 shown in FIG. 2, so that detailed descriptions thereof will be omitted.

The sensing unit 230 according to one embodiment of the present disclosure may further include a PPG sensor, a blood pressure sensor, a body temperature sensor, and/or the like, via which various types of bio-information and status information for the user may be acquired.

The communication interface 240 according to one embodiment of the present disclosure may be understood as a constituent unit that transmits and receives data via any type of known wired/wireless communication system. For example, the communication interface 240 may perform data transmission and reception by using a wired/wireless communication system such as a local area network (LAN), a wideband code division multiple access (WCDMA) network, a long term evolution (LTE) network, the wireless broadband Internet (WiBro), a 5th generation mobile communication (5G) network, an ultra-wideband wireless communication network, a ZigBee network, a radio frequency (RF) communication network, a wireless LAN, a wireless fidelity network, a near field communication (NFC) network, a Bluetooth network, or the like. Since the above-described communication systems are only examples, the wired/wireless communication system for the data transmission and reception of the communication interface 240 may be applied in various manners other than the above-described examples.

The communication interface 240 may receive the data required for the processor 110 to perform operations through wired/wireless communication with any system, any client, or the like. Furthermore, the communication interface 240 may transmit the data generated through the operations of the processor 110 through wired/wireless communication with any system, any client, or the like. For example, the communication interface 240 may receive medical data through communication with a database within a hospital environment, a cloud server for performing tasks such as the standardization of medical data, a computing device, or the like. The communication interface 240 may transmit the output data of the neural network model 10, the intermediate data derived from the computational process of the processor 110, and processed data through communication with the above-described database, server, or computing device, or the like. As an example, the processor 110 may acquire the pre-trained neural network model 10 from an external computing device (e.g., an external server device) via the communication interface 240. Furthermore, the processor 110 may acquire the training data of the neural network model 10 from an external computing device (e.g., a biosignal measurement device) via the communication interface 240.

The display 250 may display various images. The images include both still images and moving images. The display 250 may output guidance information based on the result of the diagnosis of the user 1. The display 250 may be implemented in the form of various types of displays, such as an liquid crystal display (LCD) panel, an organic light emitting diode (OLED) display, a liquid crystal on silicon (LCOS) display, a digital light processing (DLP) display, and the like. Furthermore, the display 250 may also include a driving circuit, a backlight unit, and the like, which can be implemented in the form of elements such as a-Si TFTs, low temperature poly silicon (LTPS) TFTs, and organic TFTs (OTFTs).

Meanwhile, the display 250 may be implemented as a touch screen in combination with a touch panel. In this case, the display 250 may function not only as an output interface configured to output images via the touch screen, but also as an input interface configured to receive touch input from the user 1.

The user interface 260 is a component used by the computing device 100 to interact with the user 1. The user interface 260 may include, but is not limited to, at least one of a touch sensor, a motion sensor, buttons, a jog dial, and switches. The processor 210 may receive a request for diagnosis and bio-information (e.g., the occupation, age, and gender) via the user interface 260.

The camera 270 acquires an image of an object near the user 1 by capturing the image of the object. More specifically, the camera 270 may capture an image of food consumed by the user 1. In this case, the processor 210 may determine whether the user 1 has eaten based on the status of the user 1 and the image of the food consumed by the user 1. For this purpose, the camera 270 may be implemented as an imaging device such as a CMOS image sensor (CIS) having a CMOS structure or a charge-coupled device (CCD) having a CCD structure. However, the camera 270 is not limited thereto, and may be implemented as camera modules having various resolutions capable of capturing subjects. Meanwhile, the camera 270 may be implemented as a depth camera (e.g., an IR depth camera), a stereo camera, an RGB camera, or the like.

The speaker 280 is a component that outputs various audio data that has undergone various processing operations, such as decoding, amplification, and noise filtering, via an audio processing unit (not shown). The speaker 280 may output various types of notification sounds or voice messages. According to one embodiment of the present disclosure, the processor 210 may convert an electrical signal received from an external device into the voice of the user 1 and output it via the speaker 280. As an example, the speaker 280 may output user (1) guidance information based on a diagnosis result as a voice message.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as single may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

1. A method of diagnosing heart disease based on an electrocardiogram signal, the method being performed by a computing device including at least one processor, the method comprising:
acquiring an electrocardiogram signal for a user; and
diagnosing heart disease of the user based on the acquired electrocardiogram signal;
wherein the diagnosing comprises:
computing a first score corresponding to the heart disease of the user by inputting the acquired electrocardiogram signal to a pre-trained neural network model;
determining whether to additionally diagnose the heart disease of the user based on the computed first score, and reacquiring an electrocardiogram signal for the user; and
additionally diagnosing the heart disease of the user based on the reacquired electrocardiogram signal.

2. The method of claim 1, wherein the reacquiring an electrocardiogram signal for the user comprises, when the computed first score is identified as being within a preset range, determining to perform an additional diagnosis for the heart disease of the user.

3. The method of claim 1, further comprising acquiring status information of the user;
wherein the reacquiring comprises, when an additional diagnosis for the heart disease of the user is determined to be performed, reacquiring an electrocardiogram signal for the user based on the acquired status information.

4. The method of claim 3, wherein:
the status information comprises medication information of the user; and
the reacquiring an electrocardiogram signal for the user based on the acquired status information comprises, when the electrocardiogram signal is identified as acquired while the user had not taken a medication and then the user is identified as having taken the medication based on medication information of the user, reacquiring the electrocardiogram signal.

5. The method of claim 3, wherein:
the status information comprises medication information of the user; and
the reacquiring an electrocardiogram signal for the user based on the acquired status information comprises, when the electrocardiogram signal is identified as acquired immediately after the user had taken a medication based on the medication information of the user, reacquiring the electrocardiogram signal after a preset period has elapsed since the user took the medication.

6. The method of claim 1, wherein the reacquiring an electrocardiogram signal for the user comprises, when an additional diagnosis for heart disease of the user is determined to be performed, repeatedly reacquiring an electrocardiogram signal for the user.

7. The method of claim 6, wherein the additionally diagnosing the heart disease of the user comprises, each time an electrocardiogram signal for the user is reacquired, repeatedly additionally diagnosing the heart disease of the user based on the reacquired electrocardiogram signal and providing an additional diagnosis result.

8. The method of claim 6, wherein the additionally diagnosing the heart disease of the user comprises:
computing a plurality of second scores corresponding to the heart disease of the user by inputting a plurality of repeatedly reacquired electrocardiogram signals to the pre-trained neural network model; and
additionally diagnosing the heart disease of the user based on the plurality of second scores.

9. The method of claim 6, wherein the additionally diagnosing the heart disease of the user comprises:
generating a first electrocardiogram signal by using average bits of the plurality of repeatedly reacquired electrocardiogram signals;
computing a second score corresponding to the heart disease of the user by inputting the generated first electrocardiogram signal to the pre-trained neural network model; and
additionally diagnosing the heart disease of the user based on the second score.

10. The method of claim 6, wherein the repeatedly reacquiring comprises monitoring status information of the user, determining a reacquisition time for an electrocardiogram signal for the user based on the status information, and repeatedly reacquiring an electrocardiogram signal for the user based on the determined reacquisition time.

11. The method of claim 3, wherein the reacquiring an electrocardiogram signal for the user based on the acquired status information comprises:
when the computed first score is identified as being within a preset range, determining whether there is a factor influencing the computed first score by analyzing the status information of the user; and
when it is determined that there is the factor, determining to perform an additional diagnosis for the heart disease of the user.

12. The method of claim 6, wherein the reacquiring an electrocardiogram signal for the user comprises, when an additional diagnosis for the heart disease of the user is determined to be performed, repeatedly reacquiring an electrocardiogram signal for the user at a preset time interval.

13. The method of claim 12, wherein the reacquiring an electrocardiogram signal for the user at a preset time interval comprises, when the user is identified as having undergone surgery within a preset period, changing the preset time interval from a first interval to a second interval shorter than the first interval, and reacquiring an electrocardiogram signal for the user at the second interval.

14. A computer program stored in a computer-readable storage medium, the computer program causing operations for diagnosing heart disease based on an electrocardiogram signal to be performed when executed by at least one processor, wherein the operations comprise operations of:
acquiring an electrocardiogram signal for a user; and
diagnosing heart disease of the user based on the acquired electrocardiogram signal;
wherein the operation of diagnosing heart disease of the user based on the acquired electrocardiogram signal comprises operations of:
computing a first score corresponding to the heart disease of the user by inputting the acquired electrocardiogram signal to a pre-trained neural network model;
determining whether to additionally diagnose the heart disease of the user based on the computed first score, and reacquiring an electrocardiogram signal for the user; and
additionally diagnosing the heart disease of the user based on the reacquired electrocardiogram signal.

15. A computing device for acquiring a user-customized neural network model that identifies bio-information, the computing device comprising:
memory including program codes;
a sensing unit configured to acquire an electrocardiogram signal of a user; and
a processor configured to acquire an electrocardiogram signal for the user via the sensing unit, and diagnose heart disease of the user based on the acquired electrocardiogram signal;
wherein the processor computes a first score corresponding to the heart disease of the user by inputting the acquired electrocardiogram signal to a pre-trained neural network model, determines whether to additionally diagnose the heart disease of the user based on the computed first score, reacquires an electrocardiogram signal for the user, and additionally diagnoses the heart disease of the user based on the reacquired electrocardiogram signal.
